(19) United States
(12) Patent Application Publication
(10) Pub. No.: US 2024/0090913 A1
(43) Pub. Date: Mar. 21, 2024

(54) AUGER-BASED BONE HARVESTING AND DELIVERY

(71) Applicant: Biogennix, LLC, Irvine, CA (US)

(72) Inventor: Mark Borden, Boulder, CO (US)

(21) Appl. No.: 18/368,386

(22) Filed: Sep. 14, 2023

Related U.S. Application Data
(60) Provisional application No. 63/407,082, filed on Sep. 15, 2022.

(51) Int. Cl. A61B 17/32; A61B 17/16; A61F 2/46

(52) U.S. Cl. CPC A61B 17/32002; A61B 17/1635; A61F 2/4601; A61B 2017/320024; A61B 2017/320064

(57) ABSTRACT

Auger-based bone harvesting and delivery may be provided by a surgical device comprising: a cannula having first and second openings disposed on respective first and second ends; an auger, disposed within the cannula, including a flighting arranged around an auger shaft (aligned concentrically to the cannula); and a holding area in communication with the first opening. Harvesting may be performed by rotating, a cutting tip against a bone to collect tissue; and conveying the tissue to a holding area via the auger (that is co-rotated with the cutting tip). Delivery may be performed by inserting the first opening of the cannula into a delivery site; providing a graft material in a holding area in communication with a second opening of the cannula into contact with the auger; rotating the auger to convey the graft material to the delivery site via the auger and cannula.

### CROSS-REFERENCES TO RELATED APPLICATIONS

The present disclosure claims the benefit of U.S. Provisional Patent Application No. 63/407,082 entitled “AUGER-BASED BONE HARVESTING AND DELIVERY” and filed on 2022 Sep. 15, which is incorporated herein by reference in its entirety

### BACKGROUND

Bone grafts are used in surgical procedures to replace missing or damaged bone tissue in order to facilitate a healing response. Bone grafts range in composition and source from natural bone harvested from the patient (autograft) or cadaver (allograft) to biocompatible materials such ceramics, biological glasses, and polymers (synthetic bone grafts) having properties that aid in the bone formation process. In the autograft category, various specialized tools exist to harvest bone tissue from a donor site and deliver it to the surgical site. Additional devices also aid in delivering bone graft materials through Minimally Invasive Surgery (MIS) techniques.

### SUMMARY

The present disclosure is generally related to an auger-based device and methods of use therefor in bone grafting. The described device may be used in either or both of tissue collection and Minimally Invasive Surgery (MIS) graft material delivery. The auger-based design allows users to harvest bone tissue using an MIS technique with greater precision and control, lower exertion of force on the patient, greater collection capacity, improved quality of bone tissue extracted, and easier conversion to delivery of extracted materials to another site, among other benefits. Additionally or alternatively, the auger-based design allows users to deliver grafting material in an MIS setting with greater precision and control, lower exertion of force on the graft, greater delivery capacity, improved quality of graft collected/delivered (e.g., reduced crushing of porous material), enhanced ability for delivery of custom graft mixtures, and easier conversion and transport of materials extracted from another site, among other benefits.

One embodiment of the present disclosure is a surgical device, comprising: a cannula, having an inner diameter, an outer diameter, a first opening disposed on a first end, and a second opening disposed on a second end; an auger, disposed within the cannula, including a flighting arranged around an auger shaft, wherein the auger shaft is aligned concentrically to the cannula; and a material holding area in communication with the first opening.

One embodiment of the present disclosure is a device, comprising: a rotatable material conveying means disposed in a cannula having a first opening and a second opening; a holding means in communication with the first opening; and a bone graft material disposed in the holding means and in contact with the rotatable material conveying means though the first opening.

One embodiment of the present disclosure is a method, comprising: rotating, in a first direction, a cutting tip against a bone target to collect bone tissue; and conveying the bone tissue from the bone target to a holding area via an auger disposed in a cannula, wherein the auger is co-rotated with the cutting tip, in a first direction on a longitudinal axis.

One embodiment of the present disclosure is a method, comprising: inserting a first opening of a cannula into a delivery site in a patient; providing a bone graft material in a holding area in communication with a second opening of the cannula into contact with an auger disposed in the cannula; rotating, in a second, opposite direction on a longitudinal axis, the auger; and conveying the bone graft material to the delivery site via the auger and cannula.

These embodiments represent two functions of the present disclosure in which tissue is harvested by rotating the auger in a first direction along a longitudinal axis, and tissue is delivered by rotating the auger in a second, opposite direction along a longitudinal axis. Alternatively, two auger designs may be used where the auger flighting design is modified to allow harvesting and delivery in with the same rotational direction. This can be accomplished by replacing the harvesting auger or entire harvesting auger/cannula assembly with a delivery auger or delivery auger/cannula assembly with an auger flighting design that is opposite the harvesting auger design.

### BRIEF DESCRIPTION OF THE DRAWINGS

The accompanying figures depict various elements of the one or more embodiments of the present disclosure, and are not considered limiting of the scope of the present disclosure.

In the Figures, some elements may be shown not to scale with other elements so as to more clearly show the details. Additionally, like reference numbers are used, where possible, to indicate like elements throughout the several Figures. Additionally, although primarily illustrated with a right-handled configuration, the various features may be mirrored across an axis to provide for a left-handed configuration.

It is contemplated that elements and features of one embodiment may be beneficially incorporated in the other embodiments without further recitation or illustration. For example, as the Figures may show alternative views and time periods, various elements shown in a first Figure may be omitted from the illustration shown in a second Figure without disclaiming the inclusion of those elements in the embodiments illustrated or discussed in relation to the second Figure.

FIGS. 1A-1C illustrate an example auger device, according to embodiments of the present disclosure.

FIG. 2 is a flowchart of a method for graft material extraction using a described surgical device, according to embodiments of the present disclosure.

FIG. 3 is a flowchart of a method for converting a surgical device from an extraction configuration to a delivery configuration, according to embodiments of the present disclosure.

FIG. 4 is a flowchart of a method for graft material delivery using a described surgical device, according to embodiments of the present disclosure.

FIGS. 5A-5H illustrate further views of example auger devices, according to embodiments of the present disclosure.

### DETAILED DESCRIPTION

The present disclosure is generally related to an auger-based device and methods of use therefor in bone grafting. The described device may be used in either or both of tissue collection and graft material delivery. The auger-based design allows users to harvest bone tissue through small surgical openings using a Minimally Invasive Surgery (MIS) technique with greater precision and control, lower exertion of force on the patient, greater collection capacity, improved quality of bone tissue extracted, and easier conversion to delivery of extracted materials to another site, among other benefits. Additionally or alternatively, the auger-based design allows users to deliver grafting material in a MIS setting with greater precision and control, lower exertion of force on the patient, greater delivery capacity, ability for delivery of custom graft mixtures, improved quality of bone tissue inserted, and easier conversion and transport of materials extracted from another site, among other benefits.

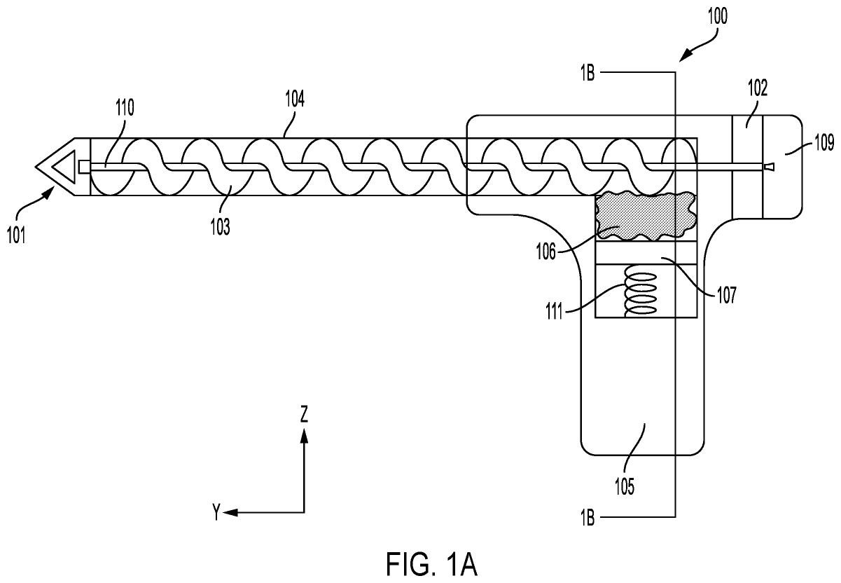

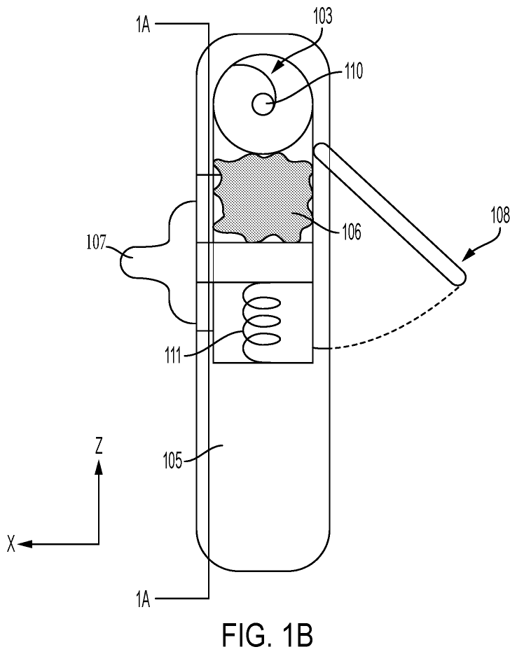

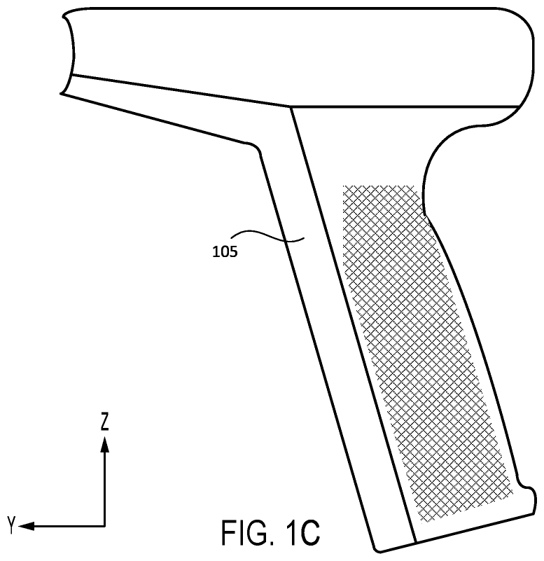

FIGS. 1A-1C illustrate an example auger device 100, according to embodiments of the present disclosure. FIG. 1A shows the auger device 100 in a cut-away side view (e.g., in a ZY plane), while FIG. 1B shows the auger device in a cut-away rear view (e.g., in a ZX plane), and FIG. 1C shows an external view of an example ergonomic handle 105 for the auger device 100.

The auger device 100 includes an auger 103 within a cannula 104, which may variously be referred to as a material conveying means. The auger 103 is positioned around and integrated to an auger shaft 110, such that the auger 103 and shaft 110 may be fabricated as a single component or fabricated separately and bonded together. The auger 103 is concentrically aligned in the cannula 104, and is sized to allow free rotation within the cannula 104. In various embodiments, the auger 103 and cannula 104 are separate devices, such that the auger 103 may be removed from the cannula 104 (and replaced with a different auger 103), or the auger 103 and cannula 104 comprise as single auger/cannula assembly, which may be attached to (and detached from) the handle 105 as a single unit.

The auger shaft 110 selectively connects to a driving means (inner knob 102 and outer knob 109) on a first end and to a cutting tip 101 on an opposing second end. Simultaneous rotation of the inner knob 102 and the outer knob 109 in one direction will harvest and move material to the material holding area 106. In some embodiments, simultaneous counter-rotation of the knobs in the opposite direction will remove material from the material holding area 106 and move material out the end of the cannula 104. Additionally, rotation of the outer knob 109 while the cutting tip 101 is held in place will unscrew the cutting tip 101 from the auger shaft 110 and allow for its removal. Additionally, or alternatively, by replacing the auger 103 (whether singly or in an assembly with the cannula 104) with a different auger 103 with flightings wound in an opposite direction, an operator can change the functionality of the device 100 from harvesting to delivery while maintaining the same operational direction for rotation. Moreover, by being able to remove or replace the auger 103, an operator is able to repair/replace dulled or fouled components and to select augers 130 with different pitch characteristics for the flightings or larger/smaller bore sizes (with associated cannulas 104).

Rotation of the auger 103 in the cannula 104 about the auger shaft 110 moves material captured by the flightings of the auger 103 from the first end towards the second end or from the second end towards the first end depending on whether the auger 103 rotates clockwise or counterclockwise relative to the longitudinal axis of the cannula 104 and the associated direction of winding for the flightings about the auger shaft 110. Accordingly, the auger device 100 may be used to harvest material from a target on the second end to deliver to a holding means on the second end, or be used to deliver material to a target on the second end from a holding means on the first end based on the direction of rotation of the auger 103.

In various embodiments, such as in FIGS. 1A and 1B, the auger 103 includes one set of flightings wound about the auger shaft 110 to define one helical path between the flightings and the inner diameter of the cannula 104. The distance between the flights may be uniform or may vary in order to modify the material movement along the path of the auger 103. In other embodiments, the auger 103 can include multiple sets of flightings to define a corresponding multitude of helical paths between adjacent flightings and the inner diameter of the cannula 104. These multiple helical paths may be communicated to different material holding area 106 to distribute extracted material among several repository areas, or to collect material from different material holding areas 106 for mixing and delivery to one delivery site.

Additionally, multiple auger designs can be employed such that harvesting, and delivery can be achieved in the same rotational direction, which is accomplished by reversing the helical path between the flightings and auger shaft from a clockwise design to a counterclockwise design (or vice versa). For example, by swapping augers 103 with different winding directions and designs for the respective flightings, an operator can use a first auger 103 designed for harvesting (e.g., the movement of tissue from the cutting tip 101 towards the holding area 106) and a second auger 103 designed for delivery (e.g., the movement of tissue from the holding chamber/holding area 106 or external material source towards a delivery site). These augers 103 can be interchanged during the surgical procedure based on user needs, preferences, or surgical conditions. Interchanging augers 103 also allows the operator to change out different designs of cutting tips 101 when interacting with different tissue types, for sharper cutting tips 101 when an initial auger 103 has dulled, or for blunted cutting tips 101 when delivering collected material to a target site.

As illustrated in FIGS. 1A and 1B, a handle 105 captures a portion of the first end of the cannula 104 and auger 103 in a cavity. The cannula 104 includes a first opening on the first end that connects to a material holding area 106 so that material extracted from a target is deposited by the auger 103 into the material holding area 106. Similarly, when the auger 103 is operated to deliver material to the second end of the cannula 104, material held in the material holding area 106 is collected by the auger 103 and moved from the opening in the first end to a second opening in the second end of the cannula 104. In various embodiments, the material holding area 106 is included in the handle 105, but may be omitted from the handle 105 (or at least one of the handles 105 in embodiments with multiple handles 105) in other embodiments.

Although illustrated with a “pistol grip” form factor, other embodiments may include other ergonomic or functional form factors for the handle 105, such as modified pistol grips as is shown in FIG. 1C (e.g., where the handle is oriented at a non-perpendicular angle relative to the longitudinal axis (in the Y direction as illustrated) of the cannula 104), T-grips (e.g., with two or more pistol style or modified pistol style grips at different arc positions around the longitudinal axis of the cannula 104), U-grips (e.g., T-grips in which at least two adjacent extensions from the cannula 104 are linked via an intervening member), mug grips (e.g., two pistol grips or modified pistol grips defined at different distances along the longitudinal axis of the cannula 140 and linked via an intermediate member), or pepper-grinder grips (e.g., the grip extends substantially evenly along the longitudinal of the cannula 104). Additionally, as illustrated in FIG. 1C, various ergonomic and friction increasing elements may be included on the handle, such as finger detents, rubberized portions, hatching, and the like.

The cannula 104 is configured for insertion into a body of a patient undergoing a bone graft procedure, and the same cannula 104 can be used for a graft material extraction portion (if performed) and the graft material delivery portion (if performed) of the bone graft procedure. Stated differently, a treating individual (e.g., a doctor, physician assistant, nurse practitioner, nurse, etc.) can extract bone tissue from a harvest site (e.g. iliac crest, tibia, or calcaneus bones) via the device 100 and use the same device 100 to deliver the extracted bone tissue to the surgical repair site. While the graft volume of previous cannulae used for bone graft material harvesting or delivery were limited by the volume of the cannulae, the material holding area 106 of the presently described device 100 allows a treating individual to advantageously harvest and/or deliver a greater volume of bone graft material than the volume of the cannula 104 used. Additionally, the cannula 104 may be repositioned after insertion into the body of the patient, allowing the treating individual to use a single-entry point into the body of the patient to harvest bone tissue from multiple sites or deliver graft material to multiple sites, thereby reducing trauma to the patient and reducing the risk of cutting blood vessels or nerves.

In some embodiments, the auger/cannula assembly may be removed from the handle 105 and replaced with a different auger/cannula assembly with a different auger design, or to replace dull, damaged, or clogged auger/cannula assemblies. In such embodiments, a harvesting auger/cannula assembly can be replaced with a delivery auger/cannula assembly. The auger/cannula assembly can be interchanged based on user needs, preferences, and surgical conditions.

In various embodiments, the cannula 104 is kept relatively small in diameter to reduce the trauma to the patient and to access smaller bone harvesting sites such as the proximal tibia or calcaneus; requiring a smaller insertion hole and reducing the risk of inadvertently cutting or touching nerves or blood vessels. Additional benefits for smaller diameter cannulae 104 compared larger diameter cannulae 104 of the same length include reduced flexion along the longitudinal axis when exerting force thereon, reduced force needed to insert or remove the cannula 104 from the body of patient, and improved rigidity or resistance against shear forces against. Additionally, the inclusion of the auger 103 in the cannula 104 provides for further rigidity against shear forces and against flexion due to longitudinal forces by supporting the inner diameter of the cannula 104. In some embodiments, the outer diameter of the cannula 104 is less than 9 millimeters (mm), which may include cannulae 104 of 7 mm, 5 mm, or 3 mm.

When used to harvest bone tissue, a cutting tip 101 is attached to the auger shaft 110 over the opening of the cannula 104, prior to insertion into the patient. The cutting tip 101 may be selectively attachable to the opening or the auger shaft 110 (e.g., via screw threads, a ball-detent, magnets, twist-locks, etc.) so that the treating individual can remove (and optionally later reattach) the cutting tip 101 to then use the cannula 104 to delivery graft material into the body of a patient with lower risk of cutting surrounding tissue to the delivery site in the body of the patient. Additionally or alternatively, the cutting tip 101 can be integrated into the auger 103, which can be detachable from the rotation knobs 102/109. In such embodiments, the integrated cutting tip 101, harvesting auger 103, and shaft 110 can be removed from the cannula 104, and be replaced with a delivery auger 103 and shaft 110 with a non-cutting tip 101, which is designed for graft delivery.

Adjustment of the auger flighting design can allow for delivery by rotating the auger 103 in the opposite direction associated with graft harvest, or in the same direction. In various embodiments, the cutting tip 101 includes at least one blade and various shapes to encourage the bone tissue cut from the extraction site to be drawn into the cannula 104 for conveyance via the auger 103 to a material holding area 106. In various embodiments, the design of the cutting tip 101 is generally conical, with windows that direct bone to the spacing between the auger flighting. Other cutting tip designs known in the art include drill bit designs, similar to those used in woodworking. The various cutting tips 101 and blades defined thereon may be collectively referred to as cutting means. In some embodiments, an outer knob 109 is included to control when the cutting tip 101 is attached or detached from the auger shaft 110 via counter rotation to unscrew the cutting tip 101 from the auger shaft 110, actuating a ball-detent to release the cutting tip 101 from the auger shaft 110, or the like.

Although not illustrated, in various embodiments, the cutting tip 101 may be absent or replaced with a nozzle or guide element that selectively connects to the auger shaft 110 or the cannula 104 in place of the cutting tip 101 for using in a delivery configuration of the device 100.

In various embodiments, the openings of the cannula 104 have different orientations and numbers based on the orientation of the cannula 104 and the one or more material holding areas 106. For example, a first opening can be arranged in a first plane perpendicular to a rotational axis of the auger 103 and the second opening is arranged in a second plane parallel to the first plane to have openings defined on the ends of an open cylindrical cannula 104. In another example, as illustrated in FIG. 1A, the first opening is defined circumferentially to the cannula and the second opening is arranged in a plane perpendicular to a rotational axis of the auger 103.

When used in a delivery configuration (e.g., with the cutting tip 101 omitted or removed), a plunger 107 in the material holding area 106 pushes the graft material into the auger 103 so that when rotated, the auger 103 collects the graft material for conveyance along the cannula 104 to the delivery site. In various embodiments, an elevator is in communication with the plunger 107 to push the plunger 107 towards the auger 103, which may include one or more of a spring 111 that decompresses to push the plunger 107, an inclined screw that rotates to elevate or retract the plunger 107, or a manual trigger for a user to set where the plunger 107 rests relative to the auger 103 (e.g., a thumb trigger of the plunger 107). The various plungers 107 and elevators may be collectively referred to as volume adjusting means, of which the illustrated spring 111 is one example.

In various embodiments, the material holding area 106 includes an access panel 108 that can selectively open or close to permit the addition or removal of material (e.g., graft) to the holding area 106. In some embodiments, the access panel 108 is part of the device 100 that slides, swings, or rotates to open or close access to the holding area 106 in addition to the opening from the cannula 104. In some embodiments, the access panel 108 is selectively attachable to the device 100 via clips, screws, hinges, or the like. In some such selectively attachable embodiments, the access panel 108 incorporates the elevator to act as an auto-feeding magazine to the auger 103. The access panel 108 therefore allows the auger-based design to not only harvest or deliver a greater volume of material than the volume of the cannula 104 (e.g., based on the volume of the holding area 106) but to exceed the combined volume of the cannula 104 and holding area 106 while remaining inserted in a patient. The various holding areas 106, material magazines, and access panels 108 may be collectively referred to as holding means.

Although illustrated in FIGS. 1A and 1B as allowing access to the holding area 106 via the ZY plane, in other embodiments, the access panel 108 may allow access from the XY plane (e.g., from the bottom of the handle 105 rather than the side) or from the opposite side from that shown in FIGS. 1A and 1B (e.g., for right-handed vs. left-handed use).

An additional benefit of the auger-based design is that conveyance of the graft material does not rely on an extruding force to push material through the cannula 104, thereby avoiding or reducing the risks of overly compressing the graft material and damaging porous structures (if present), avoiding or reducing the risks of jamming (or blowing out) the cannula 104, and reducing the forces exerted on the patient (or the treating individual). Instead, the rotation of the auger 103 within the cannula 104 conveys material in a helical path defined between the flightings of the auger 103 and the inner diameter of the cannula 104 between the target-side and collection-area side of the device 100 using a rotational force. In various embodiments, this rotational force may be imparted manually by a treating individual or mechanically (e.g., electrically or pneumatically) by a rotor device. In some embodiments, the auger shaft 110 is secured in or terminates in a bit for engaging the rotor device or a rotational inner knob 102 and outer knob 109 with a greater diameter than the auger shaft 110 or the outer diameter of the cannula 104 to provide a treating individual or rotor device with a mechanical advantage to apply torque to the auger 103 and/or cutting tip 101.

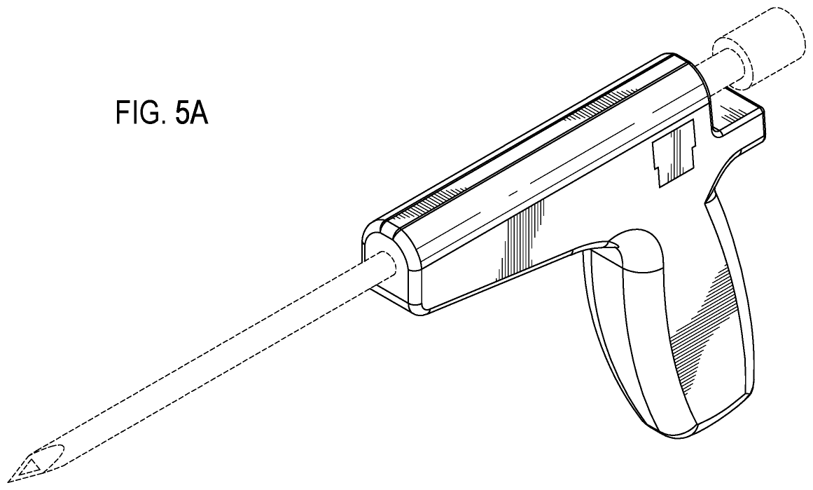

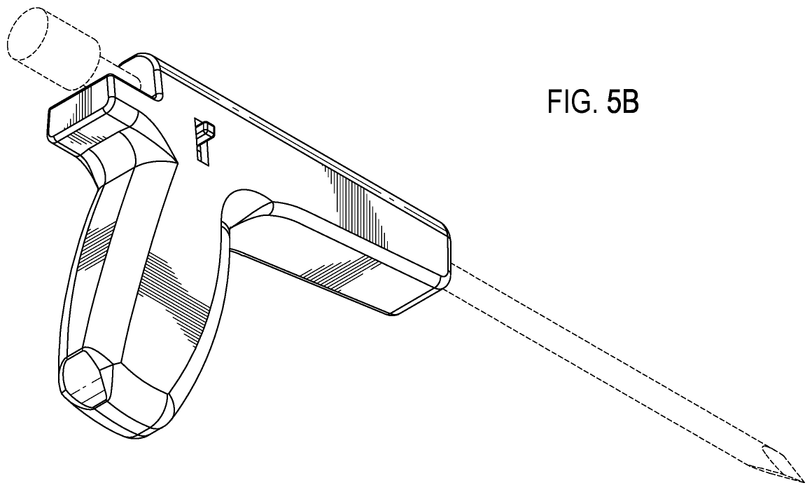

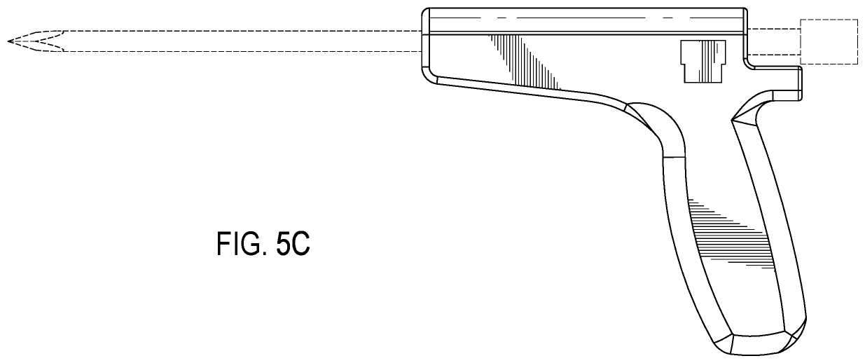

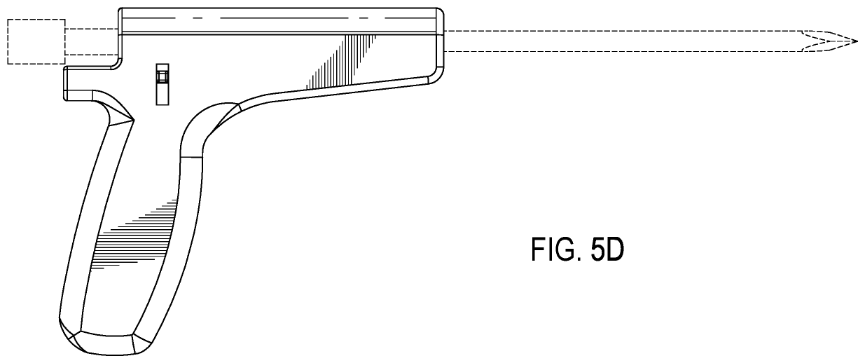

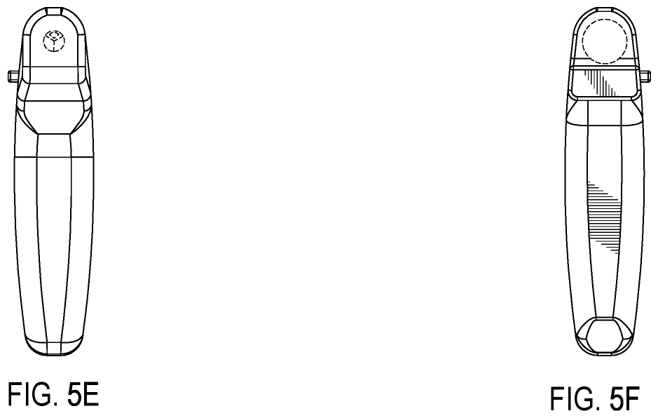

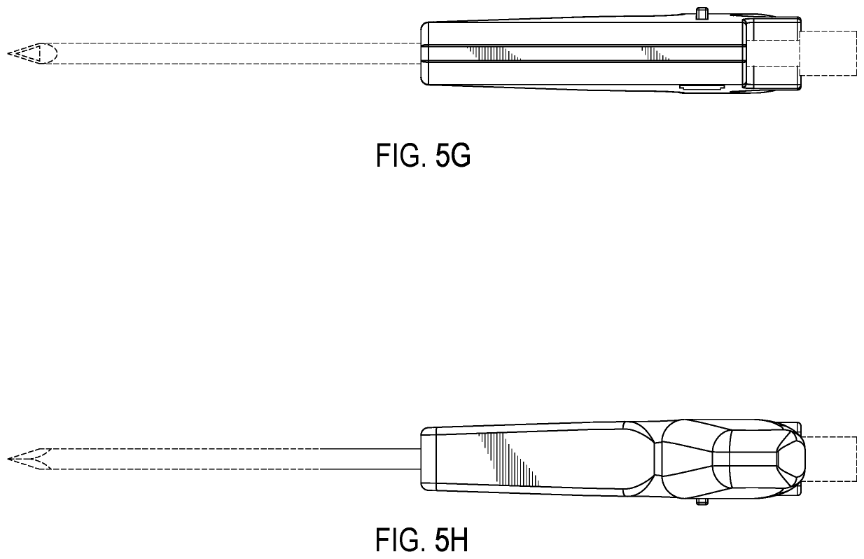

Various further views of example auger devices 100, according to embodiments of the present disclosure, are illustrated in FIGS. 5A-5H.

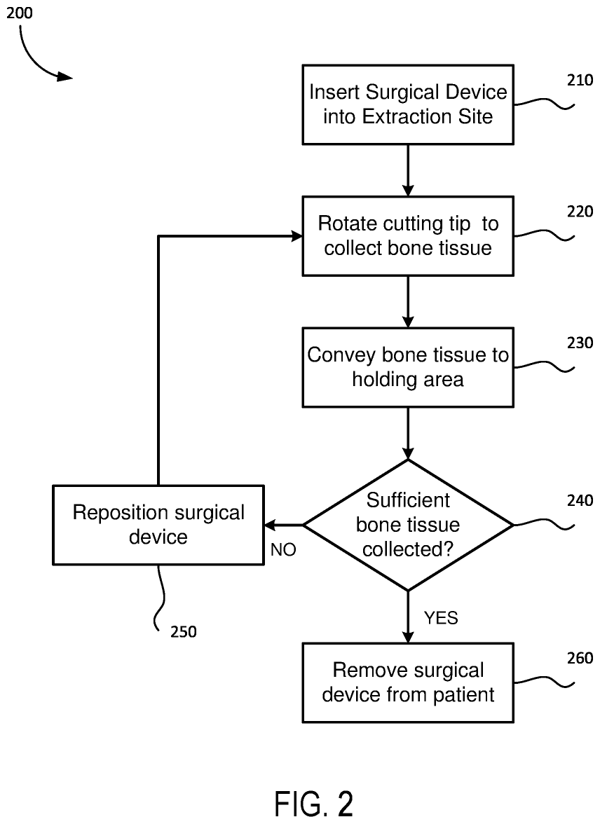

FIG. 2 is a flowchart of a method 200 for graft material extraction using a described surgical device, according to embodiments of the present disclosure. Method 200 begins at block 210, where a surgical device is inserted into autograft harvest site in a patient's body. Various extraction sites are known to practitioners for collecting bone tissue as part of MIS techniques, which include the iliac crest, the tibia, the calcaneus, and other easily accessible bones from which sufficient bone tissue can be extracted with no or minimal effect on the structural integrity of the bone.

At block 220, the operator rotates, in a first direction, a cutting tip of the surgical device against a bone target to collect bone tissue. The cutting tip is located at one end of a cannula that includes an auger disposed therein and as the cutting tip rotates and collects bone tissue, it feeds the collected bone tissue to the auger, which is simultaneously rotating with the cutting tip. In various embodiments, block 220 is continuously performed during the remainder of method 200, or may be paused at various times to pause extraction of bone tissue from the patient (e.g., to reposition the cannula per block 250, to remove the cannula from the patient per block 260, or to remove graft material from the holding area while the cannula remains in the patient).

At block 230, the auger disposed in a cannula conveys the bone tissue from the bone target to a material holding area. Because the auger is connected to the cutting tip, when the cutting tip rotates, the auger co-rotates with the cutting tip. This rotational motion progresses the collected bone material from the end of the cannula inserted into the patient to holding area located outside of the patient, which allows the surgical device to collect a greater volume of bone tissue than the volume of the cannula, with less force exerted on the patient, and with a lower risk of crushing or compacting the collected bone tissue than surgical devices that extrude or push collected bone tissue through a cannula.

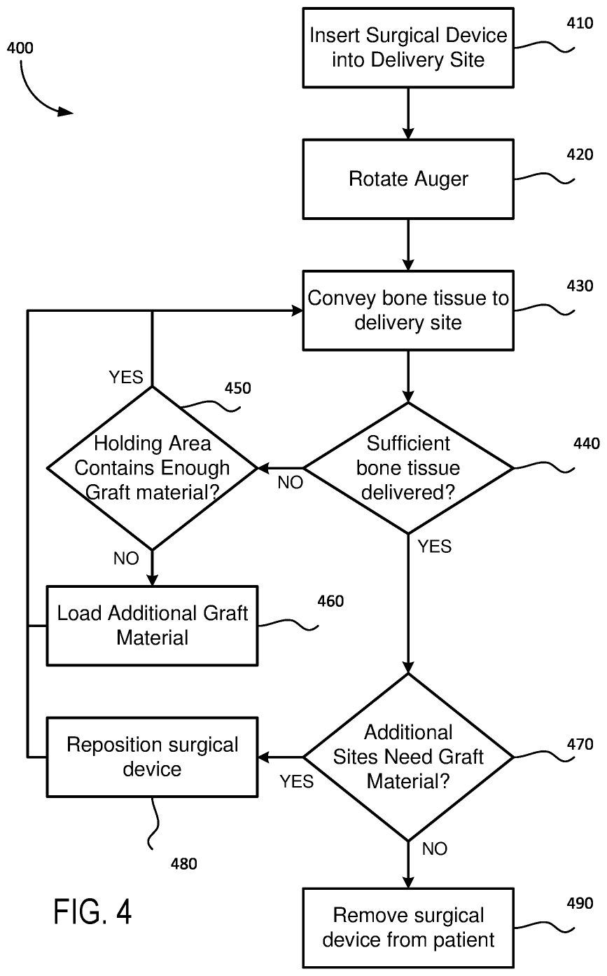

At block 240, the operator determines whether sufficient bone tissue has been collected from the bone currently being extracted from. Sufficient bone tissue may be a predefined amount specified by the operator to perform a later bone graft procedure (as per method 400 discussed in relation to FIG. 4), a volume that fills the cannula 104 and/or holding area 106, or an amount that, if exceeded, would compromise the integrity of the bone from which the material is being extracted. When the operator determines that sufficient bone tissue has been collected, method 200 proceeds to block 260. Otherwise, method 200 proceeds to block 250.

At block 250, the operator repositions the surgical device within the same surgical opening used per block 210 (to insert the cannula into the first extraction site for the patient) to position the cutting tip at a different extraction site in the same bone. For example, the operator may fully or partially extract the cannula and push the cannula back into the patient at a different angle to access a new extraction site in the same bone, without having to create a new access point in the patient. Method 200 then returns to block 220 to collect additional bone tissue from the new extraction site.

At block 260, the operator removes the surgical device from the access point in the patient. Method 200 may then conclude, iterate again at a new bone harvest site (e.g., at insertion in a new bone), or iterate again at the same or a new bone harvest site after the operator has replaced one or more components of the surgical device. For example, an operator may replace an auger 103, cutting tip 101, or a combined auger/cannula assembly mid-operation when dulled or fouled, before resuming the operation.

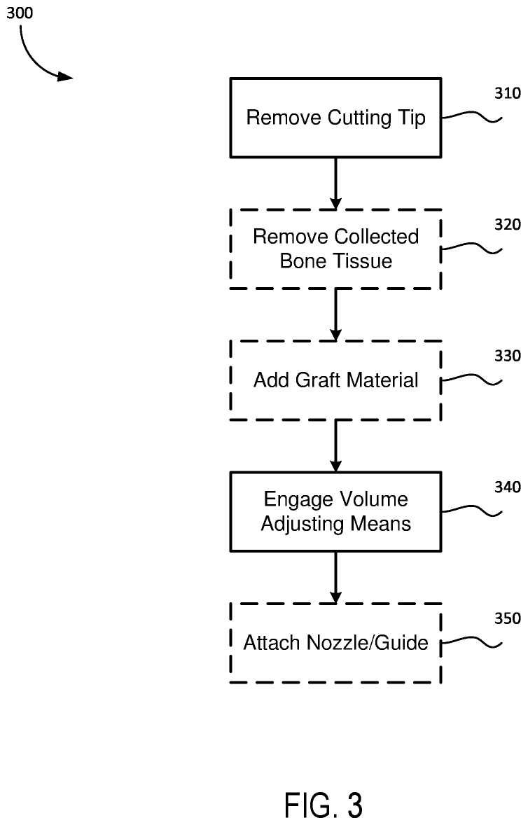

FIG. 3 is a flowchart of an example method 300 for converting a surgical device from an extraction configuration to a delivery configuration, according to embodiments of the present disclosure. In various embodiments, method 300 may begin after the conclusion of method 200 described in relation to FIG. 2.

Method 300 begins at block 310, where an operator removes a cutting tip from the cannula for a surgical device. In various embodiments, the operator may remove the cutting tip by counter rotating the cutting tip and/or the outer knob (e.g., to unscrew the cutting tip in a direction opposite to the cutting rotational direction), disengage a ball detent, twist the cutting tip to given position to align tabs of a twist-lock mechanism to disengage the cutting tip from the auger shaft, or the like.

At block 320, the operator (optionally) removes the collected bone tissue from the holding area. This can be done to allow the operator to combine the harvested autograft with supplemental graft materials such as allografts, synthetic bone grafts, bone marrow aspirate, or the like. Alternatively, when block 320 is omitted, the operator permits the collected bone tissue to remain in the holding area, and may therefore omit block 330 if no supplemental graft material is added.

At block 330, the operator (optionally) adds graft material to the holding area of the surgical device. In various embodiments, the graft material includes the collected bone tissue and optionally includes supplemental graft material such as allograft, synthetic bone graft, bone marrow aspirate, and/or other graft materials. When including multiple graft materials, the operator may include each material in different holding area (e.g., to be mixed by the auger), or as a premix in the same holding area. When using a premix, the operator may mix the graft material in the holding area or externally to the holding area. For example, an operator may remove the collected bone tissue (e.g., per block 320) and mix in a synthetic graft material until a desired volume or collected/supplemental material ratio is achieved, at which time the operator adds the mixture back to the holding area.

At block 340, the operator engages the volume adjusting means for the holding area. In various embodiments, the operator disengages a locking mechanism or otherwise permits the plunger to push the graft material in the holding area into contact with the auger.

At block 350, the operator (optionally) attaches a nozzle or guide element to the end of the cannula to which the cutting tip was previously attached. In various embodiments, the nozzle or guide element is attached using the same threads, ball-detent, or other engagement feature that connected the cutting tip to the surgical device, and allows the operator to protect the patient from rotating elements (like the auger) becoming caught on tissue at a delivery site, to focus where graft material is delivered, and the like.

Method 300 may then conclude.

FIG. 4 is a flowchart of a method 400 for graft material delivery using a described surgical device, according to embodiments of the present disclosure. In various embodiments, method 400 may begin after the conclusion of method 300 described in relation to FIG. 3. Method 400 begins a block 410, where the operator inserts the cannula of the surgical device into a surgical site in a patient. In various embodiments, the surgical site includes an intra-disk space between vertebrae of a patient (e.g., spinal fusion procedures), the site of a broken bone (e.g., trauma procedures), or any other site in which bone graft materials are to be delivered into the body of a patient.

At block 420, the operator rotates an auger included in the cannula in a second direction (opposite to the direction used to harvest bone tissue via the surgical device, such as in block 220 of method 200 discussed in FIG. 2). In various embodiments, block 420 is continuously performed during the remainder of method 400, or may be paused at various times to pause delivery of the graft material (e.g., to load additional graft material into the holding area per block 460 or reposition the cannula per block 470).

At block 430, the auger disposed in a cannula conveys the graft material from the holding area along the length of the cannula and to the delivery site. When the auger rotates, the rotational motion progresses the graft material from the holding area located outside of the patient to the end of the cannula inserted into the patient, which allows the surgical device to deposit a greater volume of graft material tissue than the volume of the cannula, with less force exerted on the patient, and with a lower risk of crushing or compacting the graft material being delivered than surgical devices that extrude or push collected bone tissue through a cannula. This is accomplished due to the helical movement path resulting from the auger design and the reduced contact of the graft with the inner cannula walls.

At block 440, the operator determines whether sufficient graft material has been delivered to the delivery site. As bone graft surgery is a procedure performed by highly trained professionals operating on individual patients with different needs based on the medical procedure being performed and variations in patient anatomy, the amount of graft material considered to be sufficient may vary, but will be readily ascertainable by the practitioner based on the facts of the operation being performed. Additionally, as some graft materials need to set or cure, “sufficient” graft material may refer to an amount of material to deposit to allow to cure before depositing another layer of graft material to the same site. For example, when graft material is placed alongside mechanically stabilizing hardware, the graft material may be placed in stages based on implantation of various hardware components. If insufficient graft material has been delivered, method 400 proceeds to block 450, otherwise, if sufficient graft material has been delivered, method 400 proceeds to block 470.

At block 450, the operator determines whether the holding area contains enough (or excess) graft material to provide sufficient graft material to the current delivery site. If the holding area contains enough (or excess) graft material, method 400 returns to block 430 to continue conveying graft material to the delivery site. Otherwise, if the holding area does not contain enough graft material, method 400 proceeds to block 460.

At block 460, the operator loads additional graft material into the holding area. Due to the location of the material holding area outside the patient, in various embodiments, the operator may load additional graft material while the cannula remains inserted in the patient, thereby allowing the total delivered volume of graft material to exceed the volume of the cannula and/or the holding area without disturbing the angle and insertion depth of the cannula in the patient. In various embodiments, the operator may pause or continue rotating the auger (per block 420) while loading the additional graft material. Method then proceeds to block 430 to continue conveying graft material to the delivery site.

At block 470, the operator determines whether an additional delivery site accessible from the access point in which the cannula is inserted into the body of the patient requires graft tissue. If the operator identifies an additional delivery site, method 400 proceeds to block 480, otherwise method 400 proceeds to block 490.

At block 480, the operator repositions the surgical device within the same surgical opening used per block 410 (to insert the cannula into the first delivery site for the patient) to position the delivery end of the cannula at a different delivery site in the body of the patient. For example, the operator may fully or partially extract the cannula and push the cannula back into the patient at a different angle to access a new delivery site, without having to create a new access point in the patient. Method 400 then returns to block 430 to continue conveying graft material to the delivery site.

At block 490, the operator removes the surgical device from the access point in the patient. Method 400 may then conclude or iterate again at another area for depositing graft material (e.g., at insertion in a new access point in the patient).

The invention may also be understood as pertaining to an MIS autograft harvesting and delivery system that can also be used with commercially available bone graft products. The device has dual functionality to harvest autograft and deliver a variety of bone graft materials. This function can be a combination of autograft harvesting and MIS delivery, or the device can solely be designed for auger-based bone graft delivery. This represents an improvement to current autograft harvesting and MIS delivery systems. In particular, current synthetic MIS bone graft systems can only deliver the bone graft product contained within a pre-loaded cannula and do not allow for mixed graft materials or intraoperative combinations with autograft. Further, current funnel systems that do allow for mixed graft materials and autograft combinations are difficult to load and often require significant force (e.g. hammering the plunger) to deliver the graft to the surgical site in the patient.

During autograft harvesting, the device is designed to harvest autograft by rotating a removable cutting tip (101) connected to an auger shaft (110). In some embodiments, this rotation is accomplished by simultaneously rotating the inner (102) and outer (109) knobs. As the bone is cut, the auger (103) within the cannula (104) rotates and moves the autograft back to the handle (105) where the autograft is stored in a graft collection area (106). The graft collection area has a spring-loaded plunger (107) that is locked down during graft collection. When locked down, the plunger (107) provides open space in the handle (105) to collect the harvested autograft material. The graft collection area (106) also has a graft access panel (108) that opens and allows the autograft to be removed from the device. This design feature allows the collected autograft to be mixed with other graft materials inside or outside the device.

During bone graft delivery, the device is also designed to function as an MIS delivery system, which can be used with the harvested autograft or various graft materials and graft material combinations (autograft, allograft, synthetics, BMP products, BMA, PRP, etc.). In one embodiment, the device is prepared for delivery by converting into an MIS delivery system by removing the cutting tip (101) and exposing the open end of the cannula (104), which may occur on the back table away from the surgical site. In some embodiments, counter screw or other release mechanism on the secondary handle [e.g., outer knob (109)] is rotated to unscrew or actuated to disengage the cutting tip (101) from the auger shaft (110). Once the cutting tip (101) is removed, the open end of the cannula (104) is exposed. In other embodiments, the device (100) is designed only for MIS delivery and a cutting tip (101) is not included. In this embodiment, cutting tip attachment/detachment mechanisms are omitted.

Once the device is ready for graft delivery or provided solely in a graft delivery form, graft material can be placed in the graft collection chamber/holding area (106). The graft access panel (108) is closed, and the spring-loaded plunger (107) is released. Once the plunger (107) is released, the spring (111) forces the contents of the graft chamber/holding area (106) up against the auger (103). Additionally, a thumb trigger of the plunger (107) on the side of the handle (105) can be pushed up to ensure the graft material stays in contact with the auger (103). To initiate delivery, the cannula tip (101) is positioned at the desired location at the graft site and the auger (103) is rotated to drive the graft material forward towards the cannula tip. Continued rotation of the auger (103) delivers the graft material from the cannula tip to the surgical site. While this is happening, continued pressure from the spring (111) and/or the thumb trigger of the plunger (107) continues to push the graft material against the auger (103). Rotation of the auger (103) is continued until the desired volume of graft material is delivered or the graft chamber/holding area (106) and auger area within the cannula (104) are emptied.

The process may be repeated until the desired amount of graft material has been delivered.

The present disclosure may also be understood with reference to the following numbered clauses.

Clause 1: A surgical device, comprising: a cannula, having an inner diameter, an outer diameter, a first opening disposed on a first end, and a second opening disposed on a second end; an auger, disposed within the cannula, including a flighting arranged around an auger shaft, wherein the auger shaft is aligned concentrically to the cannula; and a material holding area in communication with the first opening.

Clause 2: The surgical device of any of clauses 1 and 3-15, wherein the first opening is arranged in a first plane perpendicular to a rotational axis of the auger and the second opening is arranged in a second plane parallel to the first plane.

Clause 3: The surgical device of any of clauses 1, 2, and 4-15, wherein the first opening is defined circumferentially to the cannula and the second opening is arranged in a plane perpendicular to a rotational axis of the auger.

Clause 4: The surgical device of any of clauses 1-3 and 5-15, wherein the auger shaft is connected to a rotational mechanism.

Clause 5: The surgical device of any of clauses 1-4 and 6-15, further comprising: a cutting tip mounted to the auger shaft and in communication with the second opening.

Clause 6: The surgical device of any of clauses 1-5 and 7-15, wherein the cutting tip is selectively attachable to the auger shaft.

Clause 7: The surgical device of any of clauses 1-6 and 8-15, further comprising: a handle in which a portion of the first end of the cannula is secured, wherein the handle includes the material holding area.

Clause 8: The surgical device of any of clauses 1-7 and 9-15, further comprising: a plunger disposed in the material holding area; and an elevator in contact with the plunger, wherein the elevator is positioned relative to the plunger and the auger to push the plunger towards the auger.

Clause 9: The surgical device of any of clauses 1-8 and 10-15, wherein the elevator includes at least one of: a spring; a manual trigger; a selective locking device to hold or release the plunger in a given position; and an inclined screw.

Clause 10: The surgical device of any of clauses 1-9 and 11-15, wherein the material holding area includes an access panel that is selectively openable to allow removal or addition of material to the material holding area.

Clause 11: The surgical device of any of clauses 1-10 and 12-15, further comprising: a material magazine that is selectively removable from the material holding area.

Clause 12: The surgical device of any of clauses 1-11 and 13-15, wherein the outer cannula diameter is less than or equal to 9 millimeters.

Clause 13: The surgical device of any of clauses 1-12, 14, and 15, wherein a capacity of the material holding area is greater than volume of the cannula.

Clause 14: The surgical device of any of clauses 1-13 and 15, further comprising a second material holding area connected to a third opening defined on first end of the cannula.

Clause 15: The surgical device of any of clauses 1-14, wherein the auger includes a second flighting that with the flighting defines a first helical path in communication with the material holding area and not in communication with the second material holding area and a second helical path in communication with the second material holding area and not in communication with the material holding area.

Clause 16: A device, comprising: a rotatable material conveying means disposed in a cannula having a first opening and a second opening; a holding means in communication with the first opening; and a bone graft material disposed in the holding means and in contact with the rotatable material conveying means though the first opening.

Clause 17: The device of any of clauses 16, 18, and 19, further comprising a cutting means disposed on the second opening via selective attachment means.

Clause 18: The device of any of clauses 16, 17, and 19, further comprising a driving means connected to the rotatable material conveying means to impart rotation to the rotatable material conveying means.

Clause 19: The device of any of clauses 16-18, further comprising a volume adjusting means disposed in the holding means to adjust a volume of the holding means.

Clause 20: A device, comprising: a surgical cannula, having a first end configured for insertion into a body of a patient; a handle capturing a second end of the surgical cannula, including a material holding area in communication an interior of the surgical cannula via an opening on the second end of the surgical cannula; and an auger, disposed within the surgical cannula, including a flighting arranged around an auger shaft in contact with an inner diameter of the surgical cannula to define a helical path between the body of the patient and the material holding area and configured to selectively move material between the body and the material holding area as the auger rotates.

Clause 21: A method, comprising: rotating, in a first direction, a cutting tip against a bone target to collect bone tissue; and conveying the bone tissue from the bone target to a holding area via an auger disposed in a cannula, wherein the auger is co-rotated with the cutting tip.

Clause 22: The method of any of clauses 21 and 23-27, further comprising: removing the cutting tip; inserting the cannula into a delivery site; rotating the auger in a second direction, opposite the first direction; and conveying material included in the holding area to the delivery site via the auger and cannula.

Clause 23: The method of any of clauses 21, 22, and 24-27, further comprising: mixing a supplemental material with the bone tissue collected in the holding area before conveying the material included in the holding area to the delivery site.

Clause 24: The method of any of clauses 21-23 and 25-27, further comprising: pushing the material included in the holding area into contact with the auger while rotating the auger in the second direction.

Clause 25: The method of any of clauses 21-24 and 26-27, further comprising: removing the cutting tip and the auger from the cannula; inserting a second auger with a second flighting design having an opposing winding direction to a first flighting design of the auger into the cannula; inserting the cannula into a delivery site; rotating the second auger in the first direction; and conveying material included in the holding area to the delivery site via the second auger and the cannula.

Clause 26: The method of any of clauses 21-25 and 27, wherein the cannula and the auger comprise a first auger/cannula assembly, the method further comprising: removing the first auger/cannula assembly from a handle; attaching, to the handle, a second auger/cannula assembly having a second auger with a second flighting design having an opposing winding direction to a first flighting design of the auger; inserting the second auger/cannula assembly into a delivery site; rotating the second auger in the first direction; and conveying material included in the holding area to the delivery site via the second auger/cannula assembly.

Clause 27: The method of any of clauses 21-26, wherein a volume of the bone tissue collected exceeds a volume of the cannula.

Clause 28: A method, comprising: inserting a first opening of a cannula into a delivery site in a patient; providing a bone graft material in a holding area in communication with a second opening of the cannula into contact with an auger disposed in the cannula; rotating, in a first direction on a longitudinal axis, the auger; and conveying the bone graft material to the delivery site via the auger and cannula.

Clause 29: The method of any of clauses 28 and 30-32, further comprising: pushing the bone graft material included in the holding area into contact with the auger while rotating the auger in the first direction.

Clause 30: The method of any of clauses 28-29, and 31-32, wherein: the bone graft material includes bone tissue extracted from a patient via the cannula when the auger is rotated in a second direction, opposite to the first direction, and a cutting tip is connected to the first opening.

Clause 31: The method of any of clauses 28-30 and 32, further comprising: reorienting the cannula to a second delivery site while remaining inserted in the patient.

Clause 32: The method of any of clauses 28-31, further comprising: wherein a volume of the bone graft material conveyed to the delivery site exceeds a volume of the cannula.

The descriptions and illustrations of one or more embodiments provided in this disclosure are intended to provide a thorough and complete disclosure the full scope of the subject matter to those of ordinary skill in the relevant art and are not intended to limit or restrict the scope of the subject matter as claimed in any way. The aspects, examples, and details provided in this disclosure are considered sufficient to convey possession and enable those of ordinary skill in the relevant art to practice the best mode of the claimed subject matter. Descriptions of structures, resources, operations, and acts considered well-known to those of ordinary skill in the relevant art may be brief or omitted to avoid obscuring lesser known or unique aspects of the subject matter of this disclosure. The claimed subject matter should not be construed as being limited to any embodiment, aspect, example, or detail provided in this disclosure unless expressly stated herein. Regardless of whether shown or described collectively or separately, the various features (both structural and methodological) are intended to be selectively included or omitted to produce an embodiment with a particular set of features. Further, any or all of the functions and acts shown or described may be performed in any order or concurrently.

Having been provided with the description and illustration of the present disclosure, one of ordinary skill in the relevant art may envision variations, modifications, and alternate embodiments falling within the spirit of the broader aspects of the general inventive concept provided in this disclosure that do not depart from the broader scope of the present disclosure.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, and A-B-C, where the sets may include one or multiple instances of a given member (e.g., A-A, A-A-A, A-A-B, A-A-B-B-C-C-C, etc.) and any ordering thereof.

As used in the present disclosure, the term “determining” encompasses a variety of actions that may include calculating, computing, processing, deriving, investigating, looking up (e.g., via a table, database, or other data structure), ascertaining, receiving (e.g., receiving information), accessing (e.g., accessing data in a memory), retrieving, resolving, selecting, choosing, establishing, and the like.

As used in the present disclosure, the terms “substantially”, “approximately”, “about”, and other relative terms encompass values within ±5% of a stated quantity, percentage, or range unless a different approximation is explicitly recited in relation to the state quantity, percentage, or range or if the context of the value indicates that a different approximation would be more appropriate. For example, a value identified as about X % may be understood to include values between 0.95*X % and 1.05*X % or between X−0.05X and X+0.05X percent, but may stop at zero or one hundred percent in various contexts. In another example, a feature described as being substantially parallel or perpendicular to another feature shall be understood to be within ±9 degrees of parallel or perpendicular. Any value stated in relative terms shall be understood to include the stated value and any range or subrange between the indicated or implicit extremes.

As used in the present disclosure, all numbers given in the examples (whether indicated as approximate or otherwise) inherently include values within the range of precision and rounding error for that number. For example, the number 4.5 shall be understood to include values from 4.45 to 4.54, while the number 4.50 shall be understood to include values from 4.495 to 4.504. Additionally, any number or range that explicitly or by context refers to an integer amount (e.g., approximately X users, between about Y and Z states), shall be understood to round downward or upward to the next integer value (e.g., X±1 users, Y−1 and Z+1 states).

The following claims are not intended to be limited to the embodiments shown herein, but are to be accorded the full scope consistent with the language of the claims. Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112(f) unless the element is expressly recited using the phrase “means for” or “step for”. All structural and functional equivalents to the elements of the various aspects described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

We claim:

1. A surgical device, comprising:
a cannula, having an inner diameter, an outer diameter, a first opening disposed on a first end, and a second opening disposed on a second end;
an auger, disposed within the cannula, including a flighting arranged around an auger shaft, wherein the auger shaft is aligned concentrically to the cannula; and
a material holding area in communication with the first opening.

2. The surgical device of claim 1, wherein the first opening is arranged in a first plane perpendicular to a rotational axis of the auger and the second opening is arranged in a second plane parallel to the first plane.

3. The surgical device of claim 1, wherein the first opening is defined circumferentially to the cannula and the second opening is arranged in a plane perpendicular to a rotational axis of the auger.

4. The surgical device of claim 1, wherein the auger shaft is connected to a rotational mechanism.

5. The surgical device of claim 1, further comprising:
a cutting tip mounted to the auger shaft and in communication with the second opening.

6. The surgical device of claim 5, wherein the cutting tip is selectively attachable to the auger shaft.

7. The surgical device of claim 1, further comprising:
a handle in which a portion of the first end of the cannula is secured, wherein the handle includes the material holding area.

8. The surgical device of claim 7, further comprising:
a plunger disposed in the material holding area; and
an elevator in contact with the plunger, wherein the elevator is positioned relative to the plunger and the auger to push the plunger towards the auger.

9. The surgical device of claim 8, wherein the elevator includes at least one of:
a spring;
a manual trigger;
a selective locking device to hold or release the plunger in a given position; and
an inclined screw.

10. The surgical device of claim 1, wherein the material holding area includes an access panel that is selectively openable to allow removal or addition of material to the material holding area.

11. The surgical device of claim 1, further comprising:
a material magazine that is selectively removable from the material holding area.

12. The surgical device of claim 1, wherein the outer diameter is less than or equal to 9 millimeters.

13. The surgical device of claim 1, wherein a capacity of the material holding area is greater than volume of the cannula.

14. The surgical device of claim 1, further comprising a second material holding area connected to a third opening defined on first end of the cannula.

15. The surgical device of claim 14, wherein the auger includes a second flighting that with the flighting defines a first helical path in communication with the material holding area and not in communication with the second material holding area and a second helical path in communication with the second material holding area and not in communication with the material holding area.

16. A method, comprising:
rotating, in a first direction, a cutting tip against a bone target to collect bone tissue; and
conveying the bone tissue from the bone target to a holding area via an auger disposed in a cannula, wherein the auger is co-rotated with the cutting tip.

17. The method of claim 16, further comprising:
removing the cutting tip;
inserting the cannula into a delivery site;
rotating the auger in a second direction, opposite the first direction; and
conveying material included in the holding area to the delivery site via the auger and the cannula.

18. The method of claim 17, further comprising, before and while conveying the material included in the holding area to the delivery site:
pushing the material included in the holding area into contact with the auger while rotating the auger in the second direction.

19. The method of claim 16, further comprising:
removing the cutting tip and the auger from the cannula;
inserting a second auger with a second flighting design having an opposing winding direction to a first flighting design of the auger into the cannula;
inserting the cannula into a delivery site;
rotating the second auger in the first direction; and
conveying material included in the holding area to the delivery site via the second auger and the cannula.

20. The method of claim 16, wherein the cannula and the auger comprise a first auger/cannula assembly, the method further comprising:
removing the first auger/cannula assembly from a handle;
attaching, to the handle, a second auger/cannula assembly having a second auger with a second flighting design having an opposing winding direction to a first flighting design of the auger;
inserting the second auger/cannula assembly into a delivery site;
rotating the second auger in the first direction; and
conveying material included in the holding area to the delivery site via the second auger/cannula assembly.

21. The method of claim 20, further comprising:
mixing a supplemental material with the bone tissue collected in the holding area before conveying the material included in the holding area to the delivery site.

22. The method of claim 16, wherein a volume of the bone tissue collected exceeds a volume of the cannula.
